(19) United States
(12) Patent Application Publication
(10) Pub. No.: US 2007/0038305 A1
(43) Pub. Date: Feb. 15, 2007

(54) Polylock insert for an artificial hip joint

(76) Inventors: Stefan Leyen, Altbach (DE); Uwe Bunz, Wolfschlugen (DE)

Correspondence Address: FULBRIGHT & JAWORSKI, LLP, 666 FIFTH AVE, NEW YORK, NY, 10103-3198, US

(21) Appl. No.: 10/549,731

(22) PCT Filed: Mar. 24, 2004
(86) PCT No.: PCT/EP04/03093

§ 371 Date: Oct. 25, 2006

(30) Foreign Application Priority Data
Mar. 25, 2003 (EP) 103 13 413.1
Mar. 6, 2004 (DE) 10 2004 011046.8

(51) Int. Cl. A61F 2/32

(52) U.S. Cl. 623/22.15

(57) ABSTRACT

The invention relates to a hip joint prosthesis comprising a ceramic inner sliding cup (1) that, on its outside, is surrounded by a plastic covering (2), for inserting into an outer metal cup (6) or for directly implanting with the aid of bone cement. A ball head, which is mounted on a shaft, articulates inside the inner sliding cup (1), and said shaft can be anchored inside the femur. In order to increase the strength with regard to tilting and turning, the exterior of the sliding cup (1) is provided with a structuring.

The invention relates to a hip joint prosthesis in accordance with the preamble of claim 1.

An artificial hip joint as a rule consists of a sliding cup which is inserted directly or by way of a plastics covering into an outer metal cup. This metal cup is implanted in the pelvic bone. The combination of sliding cup and plastics covering is also referred to as a sandwich insert.

In this connection, a shaft, on which a ball head is arranged that articulates in the sliding cup, is implanted in the femur.

Time and again in artificial hip joints a situation can come about where the ball head shaft strikes against the acetabulum. If the impact forces are sufficiently great, this can result in the mechanical acetabulum bond breaking up. In particular, sandwich socket systems are at risk here, since the PE (polyethylene) that is predominantly used can only put up insufficient resistance against these impact forces.

Sandwich inserts are produced in various ways.

In one system, the ceramic sliding cup or the insert, as the case may be, is extrusion-coated with plastics material, with recesses being arranged on the sliding cup. The inferior polyethylene (PE) properties that result on account of the heating of the same are disadvantageous here. Furthermore, a thermal shock occurs for the ceramic sliding cup. In addition to the outlay on extrusion-coating on account of the extrusion die and the handling of the hot portions, the large amount of construction space that is required is disadvantageous.

In an alternative system, the sliding cup is anchored in the plastics covering by means of conical clamping, with in part low strength levels of the composite component. The disadvantage here is also the large amount of construction space that is required.

Preferably, the process of pressing the sliding cup into the plastics covering in the warm state is also used. In this connection, however, strength levels of the composite component that are in part too low occur. Moreover, attention is to be paid to exacting tolerances on account of the press-connection.

The underlying object of the invention is to improve a hip joint prosthesis in accordance with the preamble of claim 1 in such a way that a high level of strength with regard to tilting is achieved, with a small amount of construction space being required.

In accordance with the invention, this object is achieved in that the sliding cup has a structuring on its outside, whereby the strength with regard to tilting or turning is substantially increased with minimum construction space being required, since the structuring necessitates almost no enlargement of the construction space. Such hip joint prostheses are also referred to as polylock inserts.

The structuring is advantageously provided with large radii in the notch base in order to minimize any notch tensions that might occur. The following possibilities of a structuring are possible:
undulating depressions or
semicircular depressions.

In this connection, the notch radius at the base should be greater than 0.5 mm in order to achieve a high level of component reliability.

The undulating depressions are preferably circumferentially arranged on the outside of the sliding cup.

The sliding cup preferably has on its outside a spherical or a stepped structural form. Spherical structural forms require very little construction space.

In an advantageous embodiment, the plastics covering embraces the sliding cup at its open end, in which case the collar of the plastics covering that rests on the upper side of the sliding cup preferably covers almost half of the upper edge.

The sandwich insert is preferably produced by pressing the sliding cup into the plastics covering.

In a preferred embodiment, the inner form of the sliding cup is arranged eccentrically in relation to the outer form of the sliding cup.

The variation with respect to the coaxiality (eccentricity) advantageously amounts to at least 0.001 mm.

Further features of the invention emerge from the figures that are described in the following, in which:

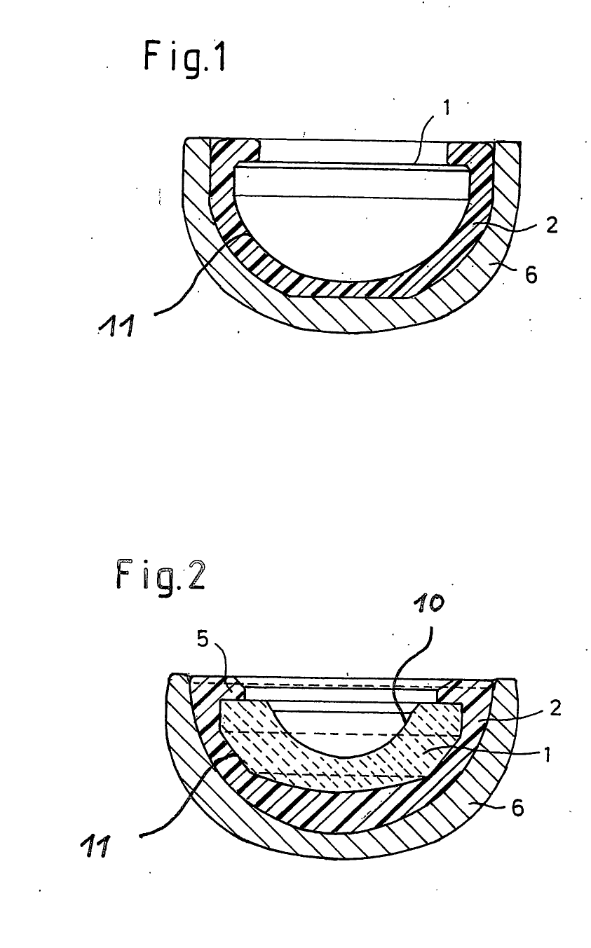

FIG. 1 shows a sandwich insert in accordance with the invention in a spherical structural form;

FIG. 2 shows a sandwich insert in accordance with the invention in a stepped structural form;

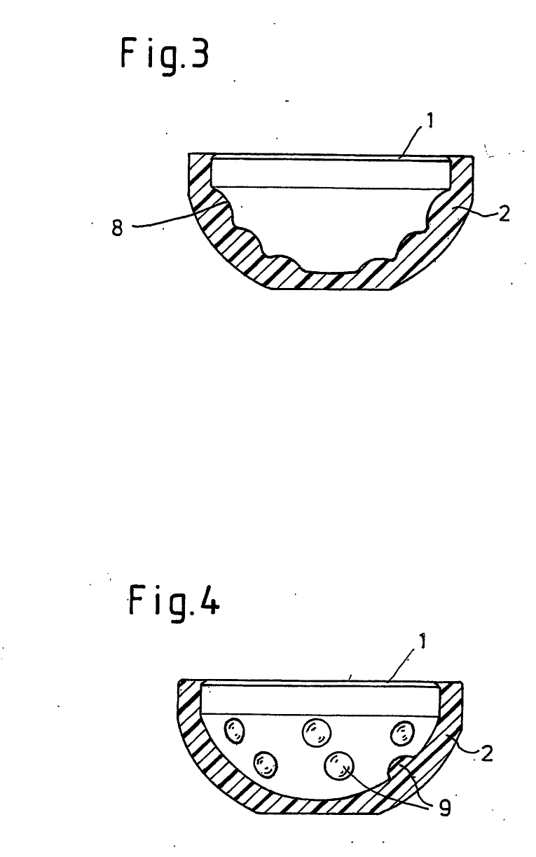

FIG. 3 shows an undulating structuring of the outside of the insert; and

FIG. 4 shows a semicircular structuring of the outside of the insert.

FIG. 1 shows a sandwich insert with a sliding cup 1 made from ceramics material in a spherical structural form. During the production, this sliding cup 1 is pressed into the plastics covering 2. The plastics covering 2 preferably consists of polyethylene (PE). This sandwich insert is inserted into an outer metal cup 6.

FIG. 2 shows an alternative embodiment with a stepped structural form of the sliding cup 1 on its outside.

At its open end the plastics covering 2 embraces the sliding cup 1, thereby improving the securement (cf. FIG. 1). The collar 5 of the plastics covering 2 that rests on the upper side of the sliding cup 1 covers almost half of the upper edge.

These two sandwich inserts shown in FIGS. 1 and 2 do not show the structuring in accordance with the invention on the outside of the sliding cup 1.

In a preferred embodiment of the invention, the inner form 10 of the sliding cup 1 is arranged eccentrically in relation to the outer form 11 of the sliding cup 1, with the variation with respect to the coaxiality (eccentricity) advantageously amounting to at least 0.001 mm.

FIG. 3 shows a sandwich insert, that is, a ceramic sliding cup 1 inserted into a plastics covering 2. Arranged on the outside of the sliding cup 1 there is a structuring which consists of, in section, an undulating depression 8. What is important in this connection is that this structuring is provided with large radii in the notch base that must amount to more than 0.5 mm. It is these radii in the notch base that first bring about good strength with regard to tilting and turning of the sliding cup 1 in the plastics covering 2.

The undulating depression 8 is circumferentially arranged on the outside of the sliding cup 1.

FIG. 4 shows a structuring that consists of semicircular depressions 9. Here as well the depressions are provided with notch radii that are greater than 0.5 mm at the notch base.

The sandwich insert, which is shown in FIGS. 1 to 4, is also referred to as a polylock insert and is inserted, for example, into an outer metal cup 6 that is then implanted into a pelvic bone. Alternatively, this insert can also be implanted directly into a pelvic bone with the aid of bone cement.

A ball head that is not shown here and preferably consists of ceramic material articulates in the sliding cup 1. This ball head is secured on a shaft that is implanted in the femur.

1-12. (canceled)

13. A hip joint prosthesis comprising an inner sliding cup made of ceramic material that is surrounded on its outside by a plastic covering, for insertion into an outer metal cup or for direct implantation with the aid of bone cement, with a ball head that is arranged on a shaft, which can be anchored in the femur, articulating in the inner sliding cup, wherein the sliding cup has a structuring on its outside.

14. A hip joint prosthesis according to claim 13, wherein the structuring is provided with large radii in the notch base.

15. A hip joint prosthesis according to claim 14, wherein the notch radius at the notch base amounts to more than 0.5 mm.

16. A hip joint prosthesis according to claim 13, wherein the structuring consists of depressions that are undulating in section.

17. A hip joint prosthesis according to claim 16, wherein the depressions that are undulating in section are circumferentially arranged on the outside of the sliding cup.

18. A hip joint prosthesis according to claim 13, wherein the structuring consists of semicircular depressions.

19. A hip joint prosthesis according to claim 13, wherein the sliding cup has on its outside a spherical or stepped structural form.

20. A hip joint prosthesis according to claim 13, wherein the plastic covering embraces the sliding cup at its pen end.

21. A hip joint prosthesis according to claim 20, wherein the collar of the plastics covering that rests on the upper side of the sliding cup covers almost half of the upper edge.

22. A hip joint prosthesis according to claim 13, wherein the sliding cup is connected to the plastics covering by being pressed into the plastics covering.

23. A hip joint prosthesis according to claim 13, wherein the inner form of the sliding cup is arranged eccentrically in relation to the outer form of the sliding cup.

24. A hip joint prosthesis according to claim 23, wherein the variation with respect to the coaxially (eccentricity) amounts to at least 0.001 mm.
